(19) United States
(12) Patent Application Publication
(10) Pub. No.: US 2008/0131394 A1
(43) Pub. Date: Jun. 5, 2008

(54) Natural Cleanser Having Antioxidant Composition

(76) Inventor: Robert Paulig, Helsinki (FI)

Correspondence Address: LADAS & PARRY LLP, 26 WEST 61ST STREET, NEW YORK, NY, 10023, US

(21) Appl. No.: 11/791,395

(22) PCT Filed: Nov. 23, 2005
(86) PCT No.: PCT/FI2005/050431

§ 371 Date: May 23, 2007

(30) Foreign Application Priority Data
Nov. 24, 2004 (FI) 20041512

(51) Int. Cl. A61K 8/97; B08B 9/08; B08B 3/08; C11D 3/382

(52) U.S. Cl. 424/74; 134/25.2; 134/40; 134/22.19; 510/405; 510/108; 510/138; 510/119; 424/725

(57) ABSTRACT

The invention relates to a soluble product extracted from coffee cherries to water, which soluble product is used either as such or concentrated as a fat-removing cleanser having antioxidant effects at the same time. Further, the product may be advantageously used as an antioxidant for zest oils of citrus fruits and along with them as cleansers and antibacterial natural products.

### FIELD OF THE INVENTION

The invention relates to a soluble product extracted from coffee cherries to water, which is used as a fat-removing cleanser. Particularly, the invention relates to a cleaning and washing agent intended for washing and cleaning cookware and ovenware as well as for cleaning ovens and grills and the like utensils that have been in contact with foodstuffs in hot conditions.

### BACKGROUND OF THE INVENTION

Generally, the basic definition of surfactants involves that part of the molecule is hydrophilic and another part is lipophilic, and therefore they may “penetrate” into two different phases simultaneously, i.e. into water and in general into grease. Surfactants reduce the surface tension significantly. The surface-active molecules orientate on the interfaces of phases and form micelles, for instance with grease, and thus they “emulsify” the grease with water away from surfaces to be washed.

The surfactants behave in different manners depending on the position of the hydrophilic group in the hydrocarbon molecule. The surfactants are often characterized by means of the HLB (hydrophilic-lipophilic balance) value. This value may vary within the range of 1 to 40, and when fats are emulsified with water the best value is between 8 and 18.

Households and restaurants clean all the time baking dishes, on which, as known, carcinogenic substances are formed when fats and proteins burn and stick to the hot surfaces. In the formulation of conventional cleaning agents this matter has been overlooked completely.

Further, in view of the fact that it is not desirable to remove all the fat from the pores of the metal ovenware (usually cast iron), but to wash away only the extra fat and protein that are stuck and chemically transformed, the problem will be seen from a totally new angle.

Moreover, many of the commercially available cleansers are irritating or at least they are not food-grade products if consumed, which would be a prerequisite for a good cleanser, because it is impossible to remove the cleanser completely from the baking dishes.

### BRIEF DESCRIPTION OF THE INVENTION

The object of the invention is to provide a cleanser by means of which the above problems can be reduced or solved. Specifically, the object of the present invention is to provide a cleanser that is suitable for cleaning cooking dishes and utensils and that is sufficiently mild.

Surprisingly it was found that the object of the invention is achieved with a solution of an aqueous coffee cherry extract.

Other uses than use as a cleanser have been described for the coffee cherry extract.

U.S. Pat. No. 1,175,490 A discloses a process for the preparation of an easily soluble dry coffee extract from coffee cherries. The extract is used as a beverage.

U.S. Pat. No. 3,576,720 A discloses a yeast growth medium, which has been prepared by extracting coffee cherry waste.

Pulgarin et al., “Comment blanchir les résidues du café noir?”, Biofutur, 1991, No. 102, p. 43-46, 48-50, CABA, CABI Publishing, UK, AN 92:4338, describe the use of wastes produced in the preparation of coffee, for instance as animal fodder, a fertilizer and a substrate in protein production.

The present invention relates to a cleanser, which is a solution separated from coffee cherries as an aqueous extract. The invention also relates to the use of this solution of an aqueous coffee cherry extract as a cleanser for removing food fats, for washing baking dishes and for washing other greasy kitchenware. The cleanser of the invention is also useful as a cosmetic substance for hand wash or as an ingredient in a shampoo. In a preferred embodiment the invention relates to a cleanser with added citrus oil.

### DETAILED DESCRIPTION OF THE INVENTION

The present invention is based on a surprising observation that an aqueous extract from coffee cherries is an excellent cleanser of dirty ovenware and other greasy surfaces.

The invention relates to a cleanser which is a solution separated as an aqueous extract from coffee cherries. The invention also relates to the use of the solution separated as an aqueous extract from coffee cherries as a food-grade cleanser for removing food fats, for washing away frying fats and for washing greasy kitchenware.

For centuries coffee cherries have been used as a foodstuff in the Arabian peninsula and in Africa. In the present invention the coffee cherry refers to a berry that originates from a plant belonging to the Coffee genus of the Rubiaceae family. The coffee cherry comprises a husk and inside the husk soft pulp that surrounds two coffee beans. The processing of the coffee cherries starts with coffee cherry picking, which is carried out by picking ripe cherries from a coffee tree by hand. The picked coffee cherries are cleaned either by washing with water or by dry sorting. From the cleaned coffee cherries the pulp is separated from the beans. This can be carried out with a so-called depulping device, for instance.

Separation with the depulping device can be performed by either a wet or a dry process. In the wet process ripe coffee cherries and water are fed into the depulping device, whereas in the dry process water is not used. When the husk and the pulp of the coffee cherries have been separated from the beans, the obtained pulp and husk are dried either in a natural manner in the sun or with the assistance of a machine at a temperature of 60 to 80° C.

Alternatively the pulp and the husk can also be treated wet.

According to a research (by Tuomas Keisteri, Department of Food Technology, University of Helsinki, 6 Feb. 2004, Master's graduate thesis, secret until 1 Dec. 2006) an aqueous extract from coffee cherries has a high antioxidant potential. The antioxidant potential generally consists of phenolic compounds and various tocopherols as well as vitamin E and vitamin C. Along with vitamin E, which is an acetate derivative of α-tocopherol, α-tocopherol is the strongest of all known antioxidants.

The invention also relates to the use of a solution separated as an aqueous extract from coffee cherries as a cosmetic substance, such as a hand wash or an ingredient in a shampoo. The cleanser containing coffee cherry extract of the invention also revitalises the skin and the hair. According to studies made at University of Helsinki (supra) the coffee cherry extract contains nearly equal amounts of antioxidant agents α-tocopherol (3.1 mg /100 dry solids) and vitamin E (3.2 mg /100 dry solids). Further, the coffee cherry extract has nearly the same capability of radical interception as red wine. In the present invention the antioxidant effect refers to prevention of oxidation, such as prevention of adverse effects of free radicals in the body, or prevention of rancidity in foodstuffs.

One embodiment of the invention relates to the use of a solution separated as an aqueous extract from the coffee cherries as an antibacterial agent. The coffee cherry is not very susceptible to bacteria or fungi, but many insects dig into it. Consequently, it seems apparent that the coffee cherry extract of the invention is antiseptic.

The cleanser of the invention can be prepared from fresh coffee cherries, but preferably it is prepared from dried coffee cherries, from which the beans have been removed and the husk and the pulp remain. The coffee cherries are pressed for removing extra water and subsequently extracted with water. The temperature of the water used is not crucial, but the extraction can be carried out at any temperature between 0 and 100° C. Preferably the extraction takes place at a temperature of 20 to 80° C., more preferably with hot water having a temperature of 50 to 55° C. The amount of extract obtained is generally about 22 to 30 percent by weight, depending on the extraction time and the number of extraction cycles.

The solution of an aqueous coffee cherry extract of the invention can be used either as such or concentrated.

The obtained extract can be concentrated by evaporation or by reverse osmosis. The reverse osmosis refers to a method, in which the flow (water) is made to pass in an opposite direction by means of external energy through a semipermeable membrane.

The cleanser of the invention has a capability of removing fats which is equal to that of citrus oils. It is known that the citrus zest oils, which are used as ecological cleansers, sensitize the human skin upon oxidation when exposed to sunlight. The strong antioxidant effect of the coffee cherry extract compensates for the oxidation tendency of the citrus oils. Thus, one embodiment of the invention relates to the use of the solution separated as an aqueous extract from the coffee cherries as an antioxidant for citrus oils. The invention also relates to a cleanser, to which citrus oil is added. Preferably, oil obtained from citrus fruit zests by steam distillation is mixed to the coffee cherry extract, most preferably lime zest oil, whereby antiviral and antibacterial properties are increased and the fragrance of the product is modified. The citrus oil is added in a suitable amount, such as 0 to 10%, preferably 0 to 2%, to provide the desired effect.

As it is commonly known, the coffee cherry extract contains 2 to 9% tannins and 3% a lignin protein compound. Both of these have both hydrophilic and lipophilic groups. Polyphenols, contained by the coffee cherry extract (4,000 mg/kg of dry coffee cherry), have an inherent capability of emulsifying fats. Tannins, in turn, form compounds directly with proteins, whereby they detach proteins from a baking tray, for instance. The coffee cherry extract of the invention is a cleanser that need not necessarily be rinsed completely away from the washed surface, unlike many other cleansers.

The cleanser of the invention cleans efficiently greasy surfaces and at the same time “neutralizes” oxidation potential remaining on the surfaces, said oxidation potential being generally considered carcinogenic.

Moreover, the cleanser of the invention is a pure natural product and does not contain sulphonated, aminated or other similar groups that provide surface activity. Nothing artificial has been added to the cleanser of the invention either.

The cleanser of the invention has an advantage of being inexpensive and effective and having a good antioxidant potential.

The cleanser of the invention has a further advantage of being economical and ecological, because the preparation thereof utilizes coffee cherry waste. Globally coffee cherries are produced every year about 3,000,000 tons, dried to the humidity of 11%, most of which is wasted, because at the coffee plantations the husk and pulp of the coffee cherries are not used but flushed in rivers.

It is apparent to a person skilled in the art that as technology progresses the basic idea of the invention can be implemented in a variety of ways. The invention and its embodiments are thus not restricted to the following example, but they may vary within the scope of the claims.

### EXAMPLE

Husk and pulp of Brazilian coffee cherries were separated from the beans by a dry method using the depulping device. About 1 kg of pulp and husk obtained from the separation was taken, pressed and extracted with 7 litres of hot water having a temperature of about 50 to 55° C. When desired, to increase the antiviral and antibacterial properties a suitable amount, such as 10 g, citrus oil was added.

1-10. (canceled)

11. A cleanser, wherein said cleanser comprises a solution separated as an aqueous extract from coffee cherries.

12. The cleanser according to claim 11, wherein said cleanser is of food-grade.

13. The cleanser according to claim 11, wherein said cleanser is prepared by concentrating an aqueous extract of coffee cherries by evaporation or by reverse osmosis.

14. The cleanser according to claim 11, wherein said cleanser further comprises citrus oil.

15. The cleanser according to claim 14, wherein said citrus oil is lime zest oil.

16. A cleaning method, wherein said method comprises the use of a solution separated as an aqueous extract from coffee cherries as a cleanser.

17. The method according to claim 16, wherein said cleanser is for removing food fats.

18. The method according to claim 16, wherein said cleanser is for washing baking dishes.

19. The method according to claim 16, wherein said cleanser is for washing kitchenware.

20. The method according to claim 16, wherein said cleanser is for hand wash or an ingredient in a shampoo.
